(19) United States
(12) Patent Application Publication
(10) Pub. No.: US 2003/0108620 A1
(43) Pub. Date: Jun. 12, 2003

(54) Pharmaceutical composition comprising a physiologically acceptable vanadium compound, salt or complex and at least a component selected from a na+/hinhibitors, cyclo-oxygenase inhibitors and caspase inhibitors

(76) Inventor: Conradus Ghosal Gho, Bunde (NL)

Correspondence Address: SUGHRUE MION, PLLC, 2100 PENNSYLVANIA AVENUE, N.W., WASHINGTON, DC, 20037, US

(21) Appl. No.: 10/168,024

(22) PCT Filed: Dec. 20, 2000
(86) PCT No.: PCT/NL00/00932

(30) Foreign Application Priority Data
Dec. 20, 1999 (WO) PCT/NL99/00789
Dec. 20, 1999 (WO) PCT/NL99/00789

(51) Int. Cl. A61K 31/555; A61K 33/26

(52) U.S. Cl. 424/646; 514/184

(57) ABSTRACT

Pharmaceutical composition for the prophylactic treatment of secondary injury of tissue, said secondary injury being induced by primary injury of mainly surrounding tissue and being the result of a traumatic event, wherein the pharmaceutical composition comprises a physiologically acceptable vanadium compound and at least a component selected from Na⁺/H⁺ exchanger inhibitors, cyclo-oxygenase inhibitors and caspase inhibitors, as active components. The pharmaceutical composition is preferably administered in a single dose and preferably before or immediately or shortly after the traumatic event.

The present invention relates to a pharmaceutical composition comprising a physiologically acceptable vanadium compound, salt or complex and at least a component selected from Na⁺/H⁺ exchanger inhibitors, cyclo-oxygenase inhibitors and caspase inhibitors. The present invention further relates to the therapeutical use of a pharmaceutical composition comprising a physiologically acceptable vanadium compound, salt or complex and and at least a component selected from Na⁺/H⁺ exchanger inhibitors, cyclo-oxygenase inhibitors and caspase inhibitors, for the prophylactic treatment of secondary injury of tissue, said secondary injury being induced by primary injury of mainly surrounding tissue, in particular surrounding tissue, and being the result of a traumatic event. The present invention relates in particular to the therapeutical use of this pharmaceutical composition for the prophylactic treatment of secondary injury of tissue, said secondary injury being induced by primary injury of mainly surrounding tissue, in particular surrounding tissue, and being the result of a traumatic event, wherein the traumatic event is ischaemia and the secondary injury is caused by reperfusion after ischaemia.

It will be clear to the person skilled in the art that secondary injury of tissue may also occur by toxins secreted by already injured tissue and that said already injured tissue has not necessarily to be located in the direct vicinity of the tissue to be protected from secondary injury. In this description the terms vanadium compound, salt or complex are used interchangeably and refer to an organic, inorganic or organometallic compound containing at least one vanadium atom and/or ion in the usual oxidation states, preferably V(II), V(III), V(IV) and/or V(V), said compound optionally being a cation or an anion and optionally being a component of an ion pair.

The use of vanadium compounds for therapeutic purposes is known. For instance, WO 90/12563 discloses the therapeutic use of compositions comprising vanadium compounds as active substance for healing mammalian tissue, e.g. the skin and organs such as heart and brain, wherein vanadium compounds are repeatedly administered in a selected concentration range over a prolonged period of time. In particular, these compositions are said to be able to prevent wrinkles in skin tissue.

The mechanism by which the vanadium compounds according to WO 90/12563 act is not clear as they may inhibit degenerative processes—reduced rate of cell death—and/or stimulate regenerative processes—increased rate of cell proliferation—wherein the net effect is that cell growth surpasses cell death which ultimately leads to healing of the injured tissue. It is, however, disclosed that the vanadium compounds have a stimulating effect on cell proliferation by inhibiting enzymatic dephosphorylation whereby growth factors such as epidermal growth factor (EGF), insulin and platelet-derived growth factor are active for a prolonged period. It is furthermore suggested that the vanadium compounds would promote the healing of e.g. heart and brain. As however heart and brain are non-proliferative tissue the suggested treatment would be unsuccessful on the basis of the postulated mechanism as set out in said cited document and a person skilled in the art would not contemplate treatment of such tissue with vanadium compounds on the basis of the cited disclosure. In this context non-proliferative tissue is understood as tissue or cells which under normal circumstances hardly proliferate. More particularly, non-proliferative tissue is only able to differentiate and thus direct or primary injury of said non-proliferative tissue, e.g. liver necrosis, can therefore not be healed by the use of a vanadium compound said to stimulate the process of proliferation. For example, the mitotoxic index of liver cells as example of non-proliferative tissue under normal conditions is extremely low and is about 1:10.000 to 1:20.000. No evidence for the promoting effect of vanadium compounds on the healing of heart or brain tissue is presented and, consequently, WO 90/12563 is considered as only disclosing the use of vanadium compounds for the enhancement of cell proliferation which in fact can only be effective in so far proliferative tissue, e.g. the skin, is involved, thereby healing direct or primary injury of said proliferative tissue. No disclosure of treatment of non-proliferative tissue is taught.

Besides the growth factor and insulin mimicking properties of vanadium compounds being known the compounds are further known to be Na/K ATP-ase inhibitors, free radical scavengers, in particular superoxide radical scavengers produced by xanthine-oxidase in injured tissues, e.g. ischaemic tissue, burns and other traumata, and inhibitors of the angiotensin II type 2 receptor.

Superoxide radicals can induce apoptosis in tissue and vanadium compounds are therefore expected to be apoptosis inhibitors by scavenging the superoxide radicals. Additionally, Yamada et al. [T. Yamada, M. Horiuchi and V. J. Dzau, Procl. Natl. Acad. Sci. U.S.A 93, 156-160 (1996)] disclose that the angiotensin II type 2 receptor mediates apoptosis. Said receptor is abundantly present in fetal tissue and immature brain and mediates anti-growth effects on vascular smooth tissue and endothelial tissue, wherein the cellular mechanism appears to involve enhancement of the dephosphorylation of mitogen-activated protein kinase (MAP kinase). It should be understood that in the present description immature brain tissue is considered as proliferative tissue. From in vitro studies they conclude that vanadate attenuated the dephosphorylation of MAP kinases thereby inhibiting the angiotensin I type 2 receptor and preventing apoptosis. Thus the suggestion of apoptosis inhibition in these types of tissue by application of vanadium compounds is provided. No data of such application is, however, provided in vitro or in vivo and no non-proliferative tissue is mentioned.

Buerke et al. [M. Buerke, T. Murohara, C. Skurk, C. Nuss, K. Tomaselli and A. M. Lefer, Proc. Natl. Acad. Sci. U.S.A. 92, 8031-8035 (1995)] disclose the use of insulin-like growth factor (IGF) for preventing reperfusion injury after ischaemia. From in vitro studies they conclude that IGF prevents myocardial injury after reperfusion and that pretreatment with IGF affords cardioprotection. Several mechanisms are presented to elucidate these effects of IGF including neutrophil accumulation in the ischaemic-reperfused myocardium, inhibition of polymorphonuclear leucocyte-induced cardiac necrosis and induction of reperfusion induced apoptosis of cardiac myocites. However, stimulation of IGF in vivo to reduce reperfusion damage, i.e. indirect or secondary injury of the myocardium, is not disclosed. In addition, Buerke et al. used IGF intracoronary as it would decompose within a very short period of time if it would be administered intravenously. Furthermore, isolated growth factors such as IGF-I, IGF-II and EGF can be obtained in usable quantities only by recombinant technology and are therefore extremely expensive. Use thereof in medicine is therefore also possible only on a very limited scale.

Olivetti et al. [G. Olivetti, R. Abbi, F. Quaini, J. Kajstura, W. Cheng, J. A. Nitahara, E. Quaini, C. DiLoreto, C. A. Beltrami, S. Krajewski, J. C. Reed and P. Anversa, N. Engl. J. Med. 16, 1131-1141 (1997)] disclose that death of myocyte cells as a result of ischaemia occurs through apoptosis and necrosis. Nothing is suggested or disclosed about indirect or secondary injury caused by reperfusion after ischaemia Nothing is mentioned concerning treatment of any kind. Nothing is disclosed about vanadium compounds.

U.S. Pat. No. 5,583,242 discloses the use of vanadium compounds to inhibit malignant B lymphocyte proliferation by inducing apoptosis in such cells. This effect could, however, not be observed in human T cell leukemia cell lines or in human colon carcinoma cells thereby indicating that vanadium compounds cannot induce apoptosis in all kinds of cells. Nothing is mentioned about other cell types. As no correlation between B cell population and injury of heart or epithelial tissue is known and B cell population does not increase in events of injury of myocardial or epithelial tissue, application of vanadium compounds for treatment of myocardial or epithelial tissue is not suggested by this document.

Furthermore, vanadium compounds, salts and complexes can be used as insulin simulators in the treatment of diabetes and for the treatment of hypertension and obesity. Known insulin-simulating vanadium salts are sodium orthovanadate (Na₃VO₄), vanadyl sulphate (VOSO₄.(H₂O)ₓ) and other reaction products of vanadate and peroxide.

WO 99/06056 (equivalent to the Dutch patent specification 1006681) discloses the use of a physiologically acceptable vanadium compounds as an active component in the preparation of a pharmaceutical composition for the prophylactic treatment of secondary injury of tissue, wherein said secondary injury is induced by primary injury of mainly surrounding tissue and wherein said secondary injury is the result of a traumatic event such as ischaemia, operations and burns on epidermal tissue.

Rohmann et al. [S. Rohmann, H. Weygandt, K -O. Minck, Eur. Heart J. 16, 1925 (1995)]disclose the use of the Na⁺/H⁺ exhanger inhibitor (3-methylsulfonyl-4-piperidinobenzoyl)-guanidine methanesulfonate (HOE 694) to reduce the infarct size in swine myocardium. They show that treatment with HOE 694 leads to a substantial reduction of the infarct size, even when HOE 694 was administered after the onset of ischaemia which implied that HOE 694 limited cell necrosis. On the other hand, the effect of HOE 694 was even more effective when administered prior to ischaemia and would therefore have cardioprotective properties. Nothing, however, is mentioned about the use of vanadium compounds in any kind of treatment.

It has been shown by Lazdunski et al. [M. Ladzunski, C. Frelin, P. Vigne, J. Mol. Cell Cardiol. 17, 1029-1042 (1985)] that during reperfusion of ischaemic myocardium the Na⁺/H⁺ exchanger is inhibited by the decreased intracellular pH occuring during ischaemia. However, it appears that the Na⁺/H⁺ exchanger is reactivated during reperfusion which results in an increase of the Na⁺ influx. The latter may lead to a dramatic increase of the intracellular Na⁺ concentration which sets in motion the exchange of Na⁺ and Ca²⁺. Thus, due to these cascade of events, the intracellular Ca²⁺ concentration increases dramatically as well and such a Ca²⁺ overload has been shown to be a crucial contributing factor in reperfusion injury (secondary injury) [M. Tani, Ann. Rev. Physiol., 52, 543-559 (1990); N. S. Dhalla, L. E. Alto, P. K. Singal, Eur. Heart J., 4 (Suppl. H), 51-56 (1983); P. A. Poole-Wilson, D. P. Harding, P. D. V. Bourdillon, M. A. Tones, J. Mol. Cell Cardiol., 16, 175-187(1984)].

WO 98/13357 discloses that the Na⁺/H⁺ exchange inhibitors of the benzo[1,4]thiazine type are effective preventives and remedies for disorders induced by intracellular acidosis at myocardial ischemia such as heart action disorders, myocardial necrosis and irregular pulse observed in ischemic heart diseases such as myocardial infarct and angina pectoris.

WO 97/25310 discloses that certain guanidine derivatives have Na⁺/H⁺ exachange inhibiting activity and can that they can be used for the treatment and/or prevention of cardiovascular diseases, arrhytmia, cerebrovascular diseases, renal diseases, arteriosclerosis, shock. They can further be used as an agent for ischemic reperfusion injury, myocardial protection, organ protection in organ transplantation and open heart surgery.

Avkiran, M., Am. J. Cardiol. 83, 10G-17G (1999) discloses that Na⁺/H⁺ inhibition may represent an effective approach to the treatment of acute myocardial ischemia in humans.

The prior art discussed above relate to only one component of the pharmaceutical composition according to the invention and does not teach or suggest a combination of a physiologically acceptable vanadium compound, salt or complex and at least a component selected from Na⁺/H⁺ exchanger inhibitors, cyclo-oxygenase inhibitors and caspase inhibitors.

Numerous vanadium compounds, salts and complexes which are effective in the treatment of diabetes, hypertension and obesity are described in U.S. Pat. No. 5,520,967. The compounds, salts and complexes concerned here are vanadium complexes of monoprotic bidentate ligands, which are capable of chelating vanadium to a five- or six-membered, unsaturated vanadium-containing ring, said ring containing at least two other hetero-atoms in addition to vanadium and said ring containing vanadium-coordinating oxygen and nitrogen hetero-atoms if the ring is a six-membered ring. Examples of compounds which form a five-membered ring as ligand are α-amino acids, hydroxamates, thiohydroxamates, α-hydroxypyridinones or α-hydroxypyrones, such as maltol or kojic acid. Examples of compounds which form a six-membered ring as ligand are substituted or unsubstituted 2-oxazolin-2-ylphenols and 2-thiazolin-2-ylphenols.

It has now been found that pharmaceutical compositions comprising a known vanadium compound, salt or complex and at least a component selected from Na⁺/H⁺ exchanger inhibitors, cyclo-oxygenase inhibitors and caspase inhibitors, is very effective in preventing indirect or secondary injury of healthy tissue, wherein said indirect or secondary injury is induced by direct or primary injury of tissue mainly surrounding the healthy tissue which is caused by a trauma. It has in particular now been found that the combination of the vanadium compound, salt or complex and at least a component selected from Na⁺/H⁺ exchanger inhibitors, cyclo-oxygenase inhibitors and caspase inhibitors, has a synergistically effect in preventing such indirect or secondary injury. The prior art discussed above, however, does neither suggest nor teach the use of the combination of vanadium compounds and at least a component selected from Na⁺/H⁺ exchanger inhibitors, cyclo-oxygenase inhibitors and caspase inhibitors to prevent indirect or secondary injury of healthy tissue which is induced by direct or primary injury of tissue mainly surrounding the healthy tissue which is a result of a traumatic event According to the invention, a combination of a vanadium compound, salt or complex and a Na⁺/H⁺ exchanger inhibitor is preferred. Even more preferred is a combination comprising a vanadium compound, salt or complex, a Na⁺/H⁺ exchanger inhibitors, a cyclo-oxygenase inhibitor and a caspase inhibitor.

The prior art suggests a link between apoptosis of some tissues but provides no in vitro data or in vivo data of inhibition of apoptosis by any treatment. It also provides no in vitro or in vivo data on treatment of indirect injury of any kind. Neither is any data provided illustrating apoptosis is responsible for indirect injury. Although the applicant does not wish to be bound by theory, it is suggested that this indirect or secondary injury of healthy tissue may be caused by apoptosis, said apoptosis being induced by directly or primarily injured cells mainly surrounding said healthy tissue. The present invention therefore relates to the use of a physiologically acceptable vanadium compound and at least a component selected from Na⁺/H⁺ exchanger inhibitors, cyclo-oxygenase inhibitors and caspase inhibitors, as active components in the preparation of a pharmaceutical composition for the prophylactic treatment of secondary injury of tissue, said secondary injury being induced by primary injury of mainly surrounding tissue and being the result of a traumatic event. As described above, it is preferred according to the invention that the pharmaceutical composition is a combination comprising a vanadium compound, salt or complex, and a Na⁺/H⁺ exchanger inhibitor. More preferably, the pharmaceutical composition comprises a combination of a vanadium compound, salt or complex, a Na⁺/H⁺ exchanger inhibitor, a cyclo-oxygenase inhibitor and a caspase inhibitor.

Without being intended to be bound by theory, it is believed that the synergistic effect of the pharmaceutical composition according to the invention is due to the involvement of different mechanistic pathways. Whereas the vanadium compound may act as an antioxidant and a growth factor and insulin simulator thereby possibly preventing apoptosis and necrosis, the Na⁺/H⁺ exchanger inhibitor prevents the formation of high intracellular Ca²⁺ concentrations which are known to be a very relevant factor in the generation of reperfusion injury. Initial experiments of the inventors have shown that the vanadium compounds as well as the Na⁺/H⁺ exchanger inhibitors afford reductions of reperfusion injury in the range of 20 to 25%, whereas the combination of a vanadium compound and a Na⁺/H⁺ exchanger inhibitor affords a reduction in the range of 35 to 50%. These higher reductions obtainable with the pharmaceutical composition according to the invention would not be predicted by the person skilled in the art on the basis available in the prior art, in particular because the in vivo effects of individual components can in general not be added together. The exact working-mechanisms of both components is not completely known as yet, and therefore the enhanced reduction of secondary injury by the combination of both components is surprising.

In general, regeneration of proliferative tissue takes place by enhanced cell proliferation when said proliferative tissue has been damaged by degeneration. On the other hand, regeneration of damaged non-proliferative tissue is obviously not possible as such tissue is not capable of proliferation.

Regeneration takes place entirely independently of the cause of the damage, whether said cause is, for example, ischaemia (infarction) or trauma. With damage as a consequence of ischaemia or trauma, indirect or secondary damage occurs in addition to direct damage or primary damage. The indirect or secondary damage occurs in tissue mainly surrounding the tissue already injured by the direct or primary damage, said indirect or secondary damage possibly being the result of a process involving apoptosis of cells of the tissue damaged by direct or primary injury. In many cases this indirect damage is greater than the direct damage. Although in proliferative tissue proliferation can be stimulated thereby inducing regeneration of said tissue, non-proliferative tissue can obviously not proliferate and the damaging of non-proliferative tissue is an irreversible process. It is therefore essential for the patient that the effects of indirect or secondary injury are restricted to a minimum or, preferably, are prevented.

Unexpectedly, very good results are obtained when the pharmaceutical preparation according to the invention is administered via a single dose, preferably intravenously, e.g. via a bolus injection, or orally. The prior art mentioning treatment with vanadium or Na⁺/H⁺ exchanger inhibitors, cyclo-oxygenase inhibitors or caspase inhibitors of any kind has been silent with regard to any advantage of such administration. The single dose in addition reduces the burden on the patient as prolonged administration which may be detrimental to the physical state of the patient or which may give rise to side effects is not required. According to the invention, the pharmaceutical composition is particularly administered intravenously.

Preferably, the pharmaceutical composition according to the invention is administered prior to the traumatic event where possible or immediately or shortly after said event. If the event involves an operation e.g. the pharmaceutical composition can be administered at a suitable moment prior to said operation. The pharmaceutical composition can be administered up to two weeks after the event, preferably within 24 h and in particular within 2 h of the event is suitable. As soon as possible after the traumatic event treatment is preferably carried out. The exact timing will depend on the circumstance of the patient and will be assessed by the attending physician.

If the traumatic event is an operation, the operation itself would cause direct or primary injury to tissue thereby causing indirect or secondary injury to mainly surrounding tissue under normal circumstances. According to the invention, said indirect or secondary injury can be prevented by administering a single dose of a suitable amount of the pharmaceutical composition according to the invention to the patient before the operation is conducted, i.e. normally within a few hours prior to the operation. Alternatively, if the traumatic event is ischaemia followed by reperfusion, the indirect or secondary injury is prevented to administer in a single dose a suitable amount of the pharmaceutical composition as fast as possible after the event took place. However, even after 24 h such an administration is capable of preventing said indirect or secondary injury. Moreover, if the traumatic event is a burn, prevention of indirect or secondary injury is achieved even if the pharmaceutical composition is administered four to five days after the burn occurred. The pharmaceutical composition can therefore be administered one week after the traumatic event, preferably within said period e.g. within 24 h and in particular 2 h after such events.

According to the invention the pharmaceutical composition according to the invention may also be added to media for tissue or organ transplantations or to media for transport of tissue and organs to be transplanted thereby preventing cell death of said tissue or organs due to secondary injury of the implanted or transplanted tissue or organ by anoxia/hypoxia or deprivation of growth factors. Also in such cases addition is preferable as soon as possible after removal of the donor organ or tissue.

According to a preferred embodiment of the present invention the tissue to be protected from indirect or secondary injury is non-proliferative tissue, in particular heart, kidney, liver, nervous or other differentiated tissue. As elucidated in the introductory part of the description directly injured non-proliferative tissue is excluded from treatment according to the invention. The non-proliferative tissue to be treated according to the invention is in particular myocardial or heart tissue. The traumatic event when treating such tissue is in particular reperfusion after ischaemia. Reperfusion damage occurs in addition to direct or primary damage caused by ischaemia and the indirect or secondary injury is very often greater than the direct or primary injury with the ratio of these injuries estimated as being 70:30. Consequently, the prevention of the secondary injury, e.g. caused by reperfusion after ischaemia, is of significant interest for a patient suffering from primary injury, e.g. caused by ischaemia.

Another important example of traumatic events to be treated according to the invention are burns on epidermal tissue. Burns, in particular second and third degree bums are known to damage structures present in the dermis such as the deep vascular plexus, the hair-adnexes (sebaceous glands) and sweat glands. The pharmaceutical compositions according to the invention when administered intravenously in a single dose are capable to protect these structures from indirect or secondary injury. These structures are non-proliferative and epidermal tissue as such is proliferative tissue. Furthermore, contractions of scars are reduced to a minimum. Interestingly, major differences were observed between wound healing effects which, on the one hand, can be influenced by growth factors or growth factor simulating agents, and, on the other, the prevention of indirect or secondary injury induced by these burns which cannot be influenced by growth factors or growth factor simulating agents only. This reiterates the comments made in the introductory part regarding prior art disclosures suggesting use of vanadium for treatment associated with stimulating growth factor activity and shows the difference between or our invention and said prior art.

Treatments of bums according to the state of the art can result in faster healing, however none are described as or are in fact capable of preventing indirect injury of structures such as the deep vascular plexus, the hair-adnexes (sebaceous glands) and sweat glands and cannot prevent severe contractions of scars. The invention, however, provides a composition which is very efficacious for preventing such indirect or secondary injury.

According to the invention the pharmaceutical compositions are preferably administered intravenously or orally. Topical treatment of burns with the pharmaceutical compositions according to the invention does not prevent secondary injury, probably due to the impermeability of the burned tissue for vanadium compounds.

Suitable vanadium salts are in principle all physiologically acceptable vanadium salts. Examples of such salts, which, for example, are already being used as an insulin replacement for diabetes patients, are sodium orthovanadate and vanadyl sulphate. Vanadium complexes that can be used are known, physiologically acceptable complexes. Said complexes comprise both vanadyl and vanadium complexes. Complex-forming units that can be used are, for example, maltol and kojic acid. According to the invention maltol, resulting in bis(maltolato)oxovanadium(IV) or the corresponding bis(maltolato)oxovanadate salt, is preferred. Both the vanadium and vanadyl salts and complexes mentioned above and other suitable vanadium and vanadyl salts and complexes are described in U.S. Pat. No. 5,583,242 and U.S. Pat. No. 5,620,967. In principle all salts and complexes mentioned in the said patents can be used.

According to the invention the vanadium compound, salt or complex is preferably an organovanadium compound, in particular bis(maltolato)oxovanadium(IV) or the corresponding bis(maltolato)oxovanadate salt.

A suitable Na⁺/H⁺ exchanger inhibitor is in particular (3-methylsulfonyl-4-piperidinobenzoyl)guanidine methanesulfonate and more in particular dimethylamiloride.

### EXAMPLE 1

Area at risk (AR) and infarcted area (IA) were determined in hearts of phenobarbital anaesthetized rats after 60 minutes coronary artery occlusion (CAO) and 180 minutes of reperfusion. At normothermia (36.5-37.5° C. body temperature), the LA/AR ratio was found to be 69±2% (mean±standard error margin, n=6) in control rats and 45±3% in rats (n=6, P<0.001) pretreated with (a) 3.3 mg/kg bodyweight (i.v. bolus in 10 minutes) bis(maltolato)oxovanadium(IV) (BMOV), (b) 3.0 mg/kg bodyweight (i.v. bolus in 10 minutes) Na⁺/H⁺ exchange inhibitor dimethylamiloride (DMA) or (c) a 1:1 combination of these two components (a) and (b) 25 minutes prior to CAO. The mean AR expressed as percentage of the left ventricle was not different between the control and the experimental group. The IA, however, which represents both direct and indirect injury (the proportion of direct injury will be equal for all groups) are tabulated below and show that the control group suffered more from indirect or secundary injury than the experimental groups. It furthermore appeared that the combination of BMOV and DMA prevents indirect injury of the myocardial tissue to a larger extent than either one of the individual components at the same dose.

| | IA (% AR) | % Reduction |
|---|---|---|
| Control | 70 | 0 |
| DMA | 55 | 21 |
| BMOV | 45 | 36 |
| GM + BMOV | 30-40 | 43-57 |

### EXAMPLE 2

The Yorkshire pig has been chosen as the experimental animal because of all animal species this domestic pig seems to be the one with morphological and functional skin characteristics nearest to human skin and so best fulfils the requirements of a model for human skin. In basic architecture it resembles human skin in the relative thickness of the epidermis and dermis, the presence of epidermal ridges, a distinct dermal papillary layer and a deep layer of subdermal fat. Compared to human the elastic fibre content of porcine dermis is relatively low, but higher than in any other species. Comparison of human and porcine epidermis and its appendages also suggests common traits. Studies on the proliferation rate of porcine epidermis show parallels with those of humans. The keratinous proteins are similar. Unlike the skin of rodents, the follicular pattern in pigs and humans is relatively sparse and arranged as single hairs or in groups of two or three follicles. Pigs do not sweat. The regulation of body temperature by the skin is more evident in humans than in the pig. In the skin of the pig no eccrine glands are found. It does have apocrine glands, but their role in thermoregulation remains debatable. The vascular anatomy of pig skin consists of a three layered network; lower, mid-dermal and sub epidermal. The size, orientation and distribution of the vessels are strikingly similar to human skin, but it does differ from humans in that the sub epidermal network is less dense. The vascularisation of the lower region of the follicle corresponds to that in humans. The healing of deep dermal bums, which depends on this phenomenon, might also be analogous. Studies on the thermal properties of porcine skin as a function of depth have been performed by measuring the tissue water content. With the use of a mathematical model the heat capacity and thermal conductivity could be calculated and results for pig skin were found to be consistent with those for human skin.

The speed of epithelialization in pigs is dependent on several factors. In full thickness wounds the epithelialization starts only from the wound margins.

In split-thickness wounds, each viable hair follicle is an islet for the reepithelialization. In split-thickness wounds of 2.2×2.2 cm, and 0.04 cm thick, in Yucatan mini pig of six months old it takes about 96 hours before complete re-epithelialization. The SD of the mean degree of epithelialization is±10%. This shows the inter individual variability. The speed of epithelialization is depending on the age, and is considerable faster in pigs weighting 7 kg in comparison of those weighting 40 kg.

There were no differences in the progression of the epithelialization in the center of the wound in comparison with the wound margin in split-thickness wounds.

Protocol 1a: Twelve identical deep burn wounds were inflicted in each animal. The animal model used is developed in the Burn Research Institute (Beverwijk, The Netherlands) and is a standard for all of the experimental burn wound research. The model is also accepted by the animal experimental commission of the University of Amsterdam.

Protocol 1b: Six biopsies of 6 mm of the burned area were taken from each big and transferred to a non-burned area. Six biopsies of 6 mm of the non-burned area were be taken from each pig and transferred to a burned area. (Determination of the indirect injury) Animals: pig, Yorkshire White, female, +/− 30 kg.

In this study we observed that structures present in the dermis such as the deep vascular plexus, the hair-adnexes (sebaceous glands) and sweat glands were protected after a cyclo-oxygenase 2 inhibitor (COX-2), a caspase inhibitor (CAS), the Na+/H+ exchange inhibitor dimethylamiloride (DMA), the bis(maltolato)oxovanadium(IV) (BMOV), a combination of DMA+BMOV or a combination of DMA+BMOV+COX-2.+CAS (equal dose in terms of g/kg bodyweight) was administered intravenously in a single dose. Also contractions of scars were considerably reduced and faster healing was observed. In the table below the percentages of preservation of the dermis is shown. These results show that at equal dose the combination of all four components COX-2, CAS, DMA and BMOV affords the best result.

| | % Preservation of dermis |
|---|---|
| Control | 0 |
| COX-2 | 10-20 |
| CAS | 10-20 |
| DMA | 20 |
| BMOV | 40-50 |
| DMA + BMOV | 50-60 |
| COX-2 + CAS + DMA + BMOV | at least 60 |

1. Pharmaceutical composition for the prophylactic treatment of secondary injury of tissue, said secondary injury being induced by primary injury of mainly surrounding tissue and being the result of a traumatic event, wherein the pharmaceutical composition comprises a physiologically acceptable vanadium compound and at least a component selected from Na⁺/H⁺ exchanger inhibitors, cyclo-oxygenase inhibitors and caspase inhibitors, as active components.

2. Pharmaceutical composition according to claim 1, wherein the composition comprises a physiologically acceptable vanadium compound and a Na⁺/H⁺ exchanger inhibitor.

3. Pharmaceutical composition according to claim 1, wherein the composition comprises a combination of a physiologically acceptable vanadium compound, a Na⁺/H⁺ exchanger inhibitor, a cyclo-oxygenase inhibitor and a caspase inhibitors.

4. Pharmaceutical composition according to any of claims 1-3, wherein the pharmaceutical composition is administered in a single dose.

5. Pharmaceutical composition according to any of the preceding claims, wherein the pharmaceutical composition is administered before or immediately or shortly after the traumatic event.

6. Pharmaceutical composition according to claim 5, wherein the pharmaceutical composition is administered within one week after the traumatic event.

7. Pharmaceutical composition according to any one of the preceding claims, wherein the pharmaceutical composition is administered intravenously or orally.

8. Pharmaceutical composition according to any one of the preceding claims, wherein the tissue is a transplant.

9. Pharmaceutical composition according to any one of the preceding claims, wherein the traumatic event is surgery.

10. Pharmaceutical composition according to any one of the preceding claims, wherein the tissue is non-proliferative tissue.

11. Pharmaceutical composition according to claim 10, wherein the non-proliferative tissue is heart, kidney, liver, nervous or other differentiated tissue.

12. Pharmaceutical composition according to claim 10 or claim 11, wherein the traumatic event is ischaemia.

13. Pharmaceutical composition according to any one of claims 1 to 9, wherein primary injury occurs to epidermal tissue.

14. Pharmaceutical composition according to any one of claims 1 to 7 and 13, wherein the traumatic event is a burn.

15. Pharmaceutical composition according to claim 14, wherein the tissue comprises a deep vascular plexus, hair-adnexes and/or sweat glands.

16. Pharmaceutical composition according to any one of the preceding claims, wherein the vanadium compound is an organovanadium compound.

17. Pharmaceutical composition according to claim 16, wherein the vanadium compound is bis(maltolato)oxovanadium(IV) or the corresponding bis(maltolato)oxovanadate salt.

18. Pharmaceutical composition according to any one of the preceding claims, werein the Na⁺/H⁺ exchange inhibitor is (3-methylsulfonyl-4-piperidinobenzoyl)guanidine methanesulfonate or dimethylamiloride.

19. Pharmaceutical composition according to any one of the preceding claims. wherein the cyclo-oxygenase-inhibitor is a cyclo-oxygenase 2 inhibitor.
